(19) United States
(12) Patent Application Publication
(10) Pub. No.: US 2006/0270739 A1
(43) Pub. Date: Nov. 30, 2006

(54) Synergistic effects of docosahexaenoic acid (DHA) and carotenoid absorption on macular pigmentation

(75) Inventors: Elizabeth J. Johnson, Boston, MA (US); D. Max Snodderly, North Augusta, SC (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: TRUSTEES OF TUFTS COLLEGE, Medford, MA (US)

(21) Appl. No.: 11/413,966

(22) Filed: Apr. 28, 2006

Related U.S. Application Data
(60) Provisional application No. 60/675,760, filed on Apr. 28, 2005.

(51) Int. Cl. A61K 31/202; A61K 31/015; A61K 31/20; A61K 31/12; A61K 31/045

(52) U.S. Cl. 514/560; 514/763; 514/690; 514/729

(57) ABSTRACT

The present invention provides compositions and methods for increasing the absorption of dietary carotenoids in humans. The methods and compositions of the invention can used to increase the concentration of retinal lutein and zeaxanthin, thereby preventing the onset and/or slowing the progression of macular degeneration. Pharmaceutical compositions comprising a therapeutically or prophylactically effective amount of a lutein and docosahexaenoic acid (DHA) are disclosed.

### RELATED APPLICATIONS

This application claims benefit of priority to U.S. Provisional Application No. 60/675,760, filed Apr. 28, 2005 entitled Synergistic Effects of Docosahexaenoic Acid (DHA) and Carotenoid Absorption on Macular Pigmentation. All contents disclosed in this applications is hereby incorporated by reference in its entirety.

### GOVERNMENT SUPPORT

Part of the work leading to this invention was carried out with United States Government support provided under a grant from the U.S. Department of Agriculture (USDA), Grant No. 581950-9-001. Therefore, the U.S. Government has certain rights in this invention.

### BACKGROUND OF THE INVENTION

Macular degeneration describes a variety of diseases that are characterized by a progressive loss of central vision associated with abnormalities of Bruch's membrane and the retinal pigment epithelium. These disorders often affect older people (age related macular degeneration (AMD)) although there are rare cases of early-onset dystrophies that may be detected in the first decade of life. AMD is the most common cause of legal blindness among individuals over the age of 60, with an incidence ranging from 11% to 18.5% in individuals over the age of 85. In the United States, AMD affects about 3.6 million individuals, with over 200,000 new cases developing per year.

There are two types of age-related macular degeneration (AMD): the dry (atrophic) form and the wet (exudative) form. The dry form of AMD affects about 90 percent of AMD patients and usually begins with the formation of tiny yellow deposits called drusen in the macula. Drusen usually do not cause serious loss of vision, but can cause distortion of vision. However, sometimes drusen will cause the macula to thin and break down, slowly leading to vision loss. The wet form of AMD occurs in about 10 percent of AMD patients. It is caused by the growth of abnormal blood vessels beneath the macula that can leak fluid and blood. The wet form of AMD typically causes significant vision problems in the affected eye and can progress very rapidly, causing permanent central vision loss. The exact cause of AMD is not known, although AMD may be hereditary.

Currently, there is no effective therapy that is capable of significantly slowing the degenerative progression of macular degeneration. Thus, today treatment is limited to invasive methods such as laser photocoagulation, which uses a high-energy laser beam to create small burns in areas of the retina with abnormal blood vessels, or photodynamic therapy, where a drug is injected into the bloodstream, concentrates in the abnormal blood vessels, and is then activated to close off the abnormal vessels.

With the increasing lifespan of people, the lack of drugs that can slow or improve AMD is becoming an acute problem. Thus, there exists a need in the art for treatments that can reduce macular degeneration.

### SUMMARY OF THE INVENTION

The present invention provides compositions and methods for increasing the absorption of dietary carotenoids in humans. The methods and compositions of the invention can used to increase the concentration of retinal lutein and zeaxanthin, thereby preventing the onset and/or slowing the progression of macular degeneration. Pharmaceutical compositions comprising a therapeutically or prophylactically effective amount of a lutein and docosahexaenoic acid (DHA) are disclosed.

In one aspect, the invention is based, in part, on the synergistic effect between docosahexaenoic acid (DHA) and carotenoids. When taken in combination with DHA, the plasma carotenoid concentration attributed to a dietary supplement is statistically higher than when taken without DHA. Alpha-carotene, β-carotene, lycopene, lutein, β-cryptoxanthin, and zeaxanthin are the predominant carotenoids found in plasma. Lutein and zeaxanthin are the predominant carotenoids found in the macula of the human eye.

In one embodiment, the invention can be used to increase the amount of retinal lutein and/or zeaxanthin. Lutein and zeaxanthin are the two carotenoids found in the macula lutea of the eye, where they have the duel functions of acting as potent antioxidants and absorbing and filtering out of harmful near-to-UV-blue light. Lutein and zeaxanthin function as antioxidants in the macula by quenching or neutralizing damaging reactive free radicals. Free radicals arise from normal biochemical reactions in the body or through exposure to toxic agents from the environment such as air pollutants or cigarette smoke. Moreover, the eye is exposed to the simultaneous presence of near-to-UV blue light and molecular oxygen, which facilitates the generation of reactive oxygen species.

In one aspect, the methods of this invention can be used prophylactically to prevent age-related macular degeneration (AMD) and/or to slow the progression of AMD. Vision loss in AMD is due to the irreversible death of photoreceptors and/or the invasion of leaky, unwanted blood vessels into the retina. Significantly lower macular pigment levels have been found in people with factors known to increase risk for AMD, such as smoking. High dietary intakes of lutein and docosahexaenoic acid (DHA) and high macular pigment (MP) can be protective against age-related macular degeneration (AMD). The methods of the invention can be used to increase the transport of lutein and/or zeaxanthin to the retina, thereby increasing the protective effect of dietary lutein and/or zeaxanthin.

The invention can be used to increase retinal carotenoid levels by increasing HDL levels. This increase in retinal carotenoid levels has a protective effect on oxidative stress that can occur in the eye. Accordingly the invention can be used to slow and/or reduce AMD. In addition, the invention can be used to increase the level of MP in the foveal region, or macula, of the retina. Lutein is a main component of MP. DHA is a key fatty acid in the retina. Lutein and zeaxanthin are transported within the blood primarily on the surface of HDL (about 53%), but also on LDL (about 31%) and VLDL (about 16%). When these lipoproteins reach retinal tissue, they are transferred to that tissue by means of lipoprotein receptors found at the surface of RPE and Muller retinal cells. HDL is a significant carrier for the retina. Within plasma, most (>60%) of apolipoprotein (Apo) E is associated with the HDL fraction. ApoE can be synthesized directly within the retina (Muller cells) and binds to receptors on ganglion cells. The subspecies of HDL containing ApoE (HDL-E) supplies lipids and lipid-soluble lutein and zeaxanthin, to the retina. Thus, by increasing HDL levels, retinal lutein and zeaxanthin levels can be concomitantly increased, which can slow and/or reduce AMD.

The present invention also provides a method of slowing the effects of aging by administering a synergistic combination of carotenoids and DHA to the subject, wherein the synergistic combination increases the absorption of the carotenoid. The present composition can slow the effects of the aging process and reduce macular degeneration.

### BRIEF DESCRIPTION OF THE FIGURES

FIG. 1 is a bar graph of the results of the verbal fluency test demonstrating the difference in the number of instances recalled for subjects given either lutein, DHA, lutein and DHA, or a placebo. For each group, significant difference from 1^{st} trial (p<): a, 0.03, b, 0.00;

FIG. 2 is a bar graph of the results of the shopping list memory test demonstrating the difference in the number of trials to learn the complete shopping list for subjects given either lutein, DHA, lutein and DHA, or a placebo. For each group, difference from 1^{st} trial (p<): a, 0.07;

FIG. 3 is a bar graph of the results of the word list memory test demonstrating the difference in the number of trials to learn the complete list for subjects given either lutein, DHA, lutein and DHA, or a placebo. For each group, difference from 1^{st} trial (p<): a, 0.07;

FIG. 4 is a bar graph of the results of the MIR apartment memory test demonstrating the difference in the number of items recalled after a delay for subjects given either lutein, DHA, lutein and DHA, or a placebo. For each group, significant difference from 1^{st} trial (p<): a, 0.02;

FIG. 5 is a graph of serum lutein concentrations in controls and subjects supplemented with lutein and/or docosahexaenoic acid (DHA), nmol/L (change from baseline), mean±se. ^{a}significantly different than baseline (p<0.05) within a group. At 2 mos, the lutein+DHA group had significantly greater increases in serum lutein all other groups (p<0.05);

FIG. 6 is a graph of serum DHA concentrations in controls and subjects supplemented with lutein and/or docosahexaenoic acid (DHA), nmol/L (change from baseline), mean±se. ^{a}significantly different than baseline (p<0.0001) within a group;

FIG. 7 is a bar graph of total macular pigment optical density (MPOD, 4 month change from baseline) in controls and subjects supplemented with lutein and/or docosahexaenoic acid (DHA), mean±se. Significantly different than baseline within a group: ^{a}p<0.0244;

FIG. 8 is a graph of macular pigment optical density (MPOD) distribution (4 month change from baseline) in controls and subjects supplemented with lutein and/or docosahexaenoic acid (DHA), mean±se. Difference from baseline within a group (p<): a, 0.03; b, 0.0906; c, 0.058, d, 0.05);

FIG. 9 is a graph of serum concentrations of HDL subfractions in subjects supplemented with lutein and/or docosahexaenoic acid (DHA (12 and 800 mg, respectively), mean±se. Significantly different than baseline within a group: a, p<0.013; b, p<0.025; c, p<0.010);

FIG. 10 is a bar graph of serum concentrations of LDL subfractions in subjects supplemented with lutein and/or docosahexaenoic acid (DHA) (12 and 800 mg, respectively), mean±se. Significantly different than baseline within a group: a, p<0.006; b, p<0.014; and

FIG. 11 is a bar graph of serum concentration of VLDL subfractions in subjects supplemented with lutein and/or docosahexaenoic acid (DHA) (12 and 800 mg, respectively), mean±se. Significantly different than baseline within a group: a, p<0.035.

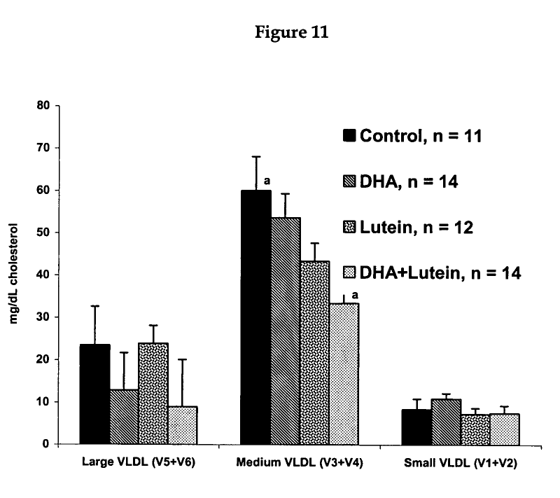

### DETAILED DESCRIPTION OF THE INVENTION

The present invention provides compositions and methods for increasing the absorption of dietary carotenoids in humans. The invention can be used to slow, reduce, or prevent the onset of neurological or memory disorders, to improve learning and cognition, and to improve memory retention and acquisition. In addition, the methods and compositions of the invention can used to increase the concentration of retinal lutein and zeaxanthin, thereby preventing the onset and/or slowing the progression of macular degeneration. Pharmaceutical compositions comprising a therapeutically or prophylactically effective amount of a lutein and docosahexaenoic acid (DHA) are disclosed.

So that the invention is more clearly understood, the following terms are defined:

The terms “neurological disorder” or “CNS disorder,” as used interchangeably herein, refer to an impairment or absence of a normal neurological function or presence of an abnormal neurological function in a subject. For example, neurological disorders can be the result of disease, injury, and/or aging. As used herein, neurological disorder also includes neurodegeneration which causes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times.

Neurological disorders include, but are not limited to, head injury, spinal cord injury, seizures, stroke, dementia, memory loss, attention deficit disorder (ADD), epilepsy, and ischemia. Neurological disorders also include neurodegenerative diseases. Neurodegeneration can occur in any area of the brain of a subject and is seen with many disorders including, but not limited to, Amyotrophic Lateral Sclerosis (ALS), multiple sclerosis, Huntington's disease, Parkinson's disease and Alzheimer's disease.

Further neurological disorders include CNS (central nervous system) damage resulting from infectious diseases such as viral encephalitis, bacterial or viral meningitis and CNS damage from tumors. The neuroprotective and/or neural regenerative strategy of the present invention can be also be used to improve the cell-based replacement therapies used to treat or prevent various demyelinating and dysmyelinating disorders, such as Pelizaeus-Merzbacher disease, multiple sclerosis, various leukodystrophies, post-traumatic demyelination, and cerebrovasuclar accidents. Disorders of the central nervous system further include mental disorders such as mood disorders (i.e., depression, bipolar disorder), anxiety disorders, memory disorders and schizophrenic disorders. In addition, the present invention may also find use in enhancing the cell-based therapies used to repair damaged spinal cord tissue following a spinal cord injury.

The term “memory disorder,” as used herein, refers to a diminished mental registration, retention or recall of past experiences, knowledge, ideas, sensations, thoughts or impressions. Memory disorder may affect short and/or long-term information retention, facility with spatial relationships, memory (rehearsal) strategies, and verbal retrieval and production. The term memory disorder is intended to include dementia, slow learning and the inability to concentrate. Common causes of a memory disorder are age, severe head trauma, brain anoxia or ischemia, alcoholic-nutritional diseases, drug intoxications, and neurodegenerative diseases. For example, a memory disorder is a common feature of neurodegenerative diseases, such as Alzheimer's disease (i.e. Alzheimer-type dementia). Memory disorders also occur with other kinds of dementia such as AIDS Dementia; Wernicke-Korsakoffs related dementia (alcohol induced dementia); age related dementia, multi-infarct dementia, a senile dementia caused by cerebrovascular deficiency, and the Lewy-body variant of Alzheimer's disease with or without association with Parkinson's disease. Creutzfeldt-Jakob disease, a spongiform encephalopathy caused by the prion protein, is a rare dementia with which memory disorder is associated. Loss of memory is also a common feature of brain-damaged patients. Non-limiting examples of causes of brain damage which may result in a memory disorder include stroke, seizure, an anaesthetic accident, ischemia, anoxia, hypoxia, cerebral edema, arteriosclerosis, hematoma or epilepsy; spinal cord cell loss; and peripheral neuropathy, head trauma, hypoglycemia, carbon monoxide poisoning, lithium intoxication, vitamin (B1, thiamine and B12) deficiency, or excessive alcohol use. Korsakoff's amnesic psychosis is a rare disorder characterized by profound memory loss and confabulation, whereby the patient invents stories to conceal his or her memory loss. It is frequently associated with excessive alcohol intake. Memory disorder may furthermore be age-associated; the ability to recall information such as names, places and words seems to decrease with increasing age. Transient memory loss may also occur in patients, suffering from a major depressive disorder, after electro-convulsive therapy.

The term “therapeutically effective amount” refers to an amount effective, at dosages and for periods of time necessary, to achieve the desired therapeutic result. A therapeutically effective amount of the supplement may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

The term “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The term “docosahexaenoic acid” or “DHA” refer to n-3 highly unsaturated fatty acids, including, but not limited to, omega-3 fatty acids such as eicosapentaenoic acid (EPA), docosahexaenoic acid (DHA), α-linolenic acid (ALA), docosapentaenoic acid (DPA), and precursors and analogs thereof. Eicosapentaenoic acid, a long-chain polyunsaturated fatty acid of the n-3 or omega-3 type, is a major component of fish oil. EPA is an all cis polyunsaturated fatty acid containing 20 carbons and 5 double bonds. EPA is also known as EPA; C20: 5n-3 and cis-5,8,11,14,17-eicosapentaenoic acid. EPA is a precursor of the series-3 prostaglandins, the series-S leukotrienes and the series-3 thromboxanes, which are anti-artherogenic and antithrombogenic. EPA is found naturally in the form of triacylglycerols (TAGs). The structural formula of EPA is as follows:

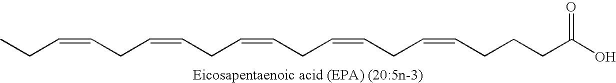

Docosahexaenoic acid, a long-chain polyunsaturated fatty acid (LCPUFA) of the n-3 or omega-3 type, is a major component of fish oil. DHA is an all cis polyunsaturated fatty acid containing 22 carbon atoms and 6 double bonds. DHA is also known as DHA; C22: 6n-3 and cis-4, 7, 10, 13, 16, 19-docosahexaenoic acid. DHA is a vital component of the phospholipids of human cellular membranes, especially those in the brain and retina. DHA occurs naturally in the form of triacylglycerols (TAGs). DHA has the following structural formula: 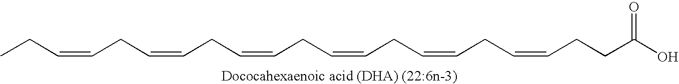
While DHA and EPA occur at high levels in fish oil and usually exist in the triglyceride form, the term DHA as used throughout this specification means not only DHA or EPA as such but also the corresponding glycerin ester (e.g. triglyceride), alkyl ester (e.g. ethyl ester), or other derivatives, and analogs thereof. For examples of EPA and DHA analogs and methods of preparation and isolation thereof see, for example, U.S. Pat. No. 6,670,396, the contents of which is hereby incorporated in its entirety.

The term “subject” as used herein refers to any living organism capable of eliciting an immune response. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The term “free radical” as used herein refers to molecules containing at least one unpaired electron. Most molecules contain even numbers of electrons, and their covalent bonds normally consist of shared electron pairs. Cleavage of such bonds produces two separate free radicals, each with an unpaired electron (in addition to any paired electrons). They may be electrically charged or neutral and are highly reactive and usually short-lived. They combine with one another or with atoms that have unpaired electrons. In reactions with intact molecules, they abstract a part to complete their own electronic structure, generating new radicals, which go on to react with other molecules. Such chain reactions are particularly important in decomposition of substances at high temperatures and in polymerization. In the body, oxidized free radicals can damage tissues. Antioxidant may reduce these effects. Heat, ultraviolet light, and ionizing radiation all generate free radicals. Free radicals are generated as a secondary effect of oxidative metabolism. An excess of free radicals can overwhelm the natural protective enzymes such as superoxide dismutase, catalase, and peroxidase. Free radicals such as hydrogen peroxide (H₂O₂), hydroxyl radical (HO∘), singlet oxygen (¹O₂), superoxide anion radical (O∘₂⁻), nitric oxide radical (NO∘), peroxyl radical (ROO∘), peroxynitrite (ONOO⁻) can be in either the lipid or compartments.

Age-Related Macular Degeneration (AMD)

Age-related losses in visual function are major health concerns in the U.S. Age-related macular degeneration (AMD) is a leading cause of blindness. Some epidemiologic reports suggest that intake of foods rich in lutein protects against AMD. Lutein, along with its stereoisomer, zeaxanthin, selectively accumulate in the retina and are particularly dense in the foveal region, or macula, where they are the main components of the macular pigment (MP). The macula is located in the posterior portion of the retina and possesses the highest concentration of cone photoreceptors responsible for central vision and high resolution visual acuity. Lutein is known to function as an antioxidant and blue light filter and thereby may protect the macula from light-initiated oxidative damage to the retina and retinal pigment epithelium. Oxidative stress is high in the eye due to the intense light exposure and the high rate of oxidative metabolism in the retina. It is generally believed that cumulative oxidative damage is responsible for aging and therefore, may play an important role in the pathogenesis of AMD. The appearance of oxidation products of lutein and zeaxanthin within the retina is consistent with the idea that these pigments can function as antioxidants in vivo. In one aspect, the present invention provides a method of altering the level of MP in a subject's eye. As shown in the Examples, the invention demonstrates that the level of MP can be manipulated in the cone-rich fovea when a subject ingests the composition of the present invention.

Lutein and Zeaxanthin

Carotenoids, naturally-occurring pigments which are synthesized by plants, algae, bacteria, and certain animals, such as birds and shellfish have antioxidant activities. Carotenoids are a group of hydrocarbons (e.g., carotenes) and their oxygenated, alcoholic derivatives (e.g., xanthophylls), and include, for example, actinioerythrol, astaxanthin, bixin, canthaxanthin, capsanthin, capsorubin, β-8′-apo-carotenal (apo-carotenal), β-12′-apo-carotenal, α-carotene, β-carotene, “carotene” (a mixture of α- and β-carotenes), γ-carotene, β-cryptoxanthin, lutein, lycopene, violerythrin, zeaxanthin, and esters of hydroxyl- or carboxyl-containing members thereof. As a result of a high intake of fruits and vegetables, 34 carotenoids and their metabolites are found in human serum and tissues at varying concentrations. Alpha-carotene, β-carotene, lycopene, lutein, β-cryptoxanthin, and zeaxanthin are the predominant carotenoids found in plasma. Lutein and zeaxanthin are the only carotenoids found in the macula of the human eye.

Lutein and zeaxanthin belong to the xanthophyll class of carotenoids, also known as oxycarotenoids, which are natural fat-soluble yellowish pigments. Lutein and zeaxanthin are are derived exclusively from dietary sources, such as dark green leafy vegetables and orange and yellow fruits and vegetables. Dietary sources of these dihydroxycarotenoids include corn, egg yolks, broccoli, green beans, green peas, brussel sprouts, cabbage, kale, collard greens, spinach, lettuce, kiwi and honeydew. The xanthophylls, which in addition to lutein and zeaxanthin, include alpha-and beta-cryptoxanthin, contain hydroxyl groups, which makes them more polar than other carotenoids. Although lutein and zeaxanthin have identical chemical formulas and are isomers, they are not stereoisomers. They are both polyisoprenoids containing 40 carbon atoms and cyclic structures at each end of their conjugated chains. As used herein, “lutein” is intended to include lutein and all its isomers, including zeaxanthin. They both occur naturally as all-trans (all-E) geometric isomers and the principal difference between them is in the location of a double bond in one of the end rings.

Lutein and zeaxanthin are concentrated in the macula of the human eye. While over 15 different dietary carotenoids are detectable in human serum, only lutein and zeaxanthin and their metabolites are found to any substantial extent in the retina. Zeaxanthin concentration is highest in the center of the fovea, whereas lutein is relatively abundant in the perifoveal region. The absorption spectra of lutein and zeaxanthin enable these macular pigments to absorb blue light, which the can damage the retina. Scattering and chromatic aberration of blue light can be minimized by these macular pigments. In addition, these carotenoids are also potent antioxidants. Therefore, zeaxanthin and lutein can protect the retina against oxidative damage in the macula where free radicals can be generated by lengthy light exposure, high oxygen tension, and high metabolic rate. Epidemiologic studies have shown that people with higher dietary or plasma lutein/zeaxanthin have reduced risk for advanced stages of AMD. In one aspect, the methods of this invention can be used to increase the absorption of dietary carotenoids, such as lutein and zeaxanthin. This increase in absorption can be used to increase the amount of lutein/zeaxanthin that can be transported to the macular. In addition, the methods of the invention can be used to increase the absorption of carotenoids, such as lutein and zeaxanthin, which can lead to an increase of the concentration of serum carotenoids, which can lead to improvements in cognitive function.

Lutein and zeaxanthin are transported within the blood primarily on the surface of HDL (about 53%), but also on LDL (about 31%) and VLDL (about 16%). When these lipoproteins reach retinal tissue, they are transferred to that tissue by means of lipoprotein receptors found at the surface of RPE and Muller retinal cells. Although the precise mechanism has not yet been established, there is increasing evidence suggesting that HDL might be the most significant carrier for the retina. For example, within the plasma most (>60%) of apolipoprotein (Apo) E is associated with the HDL fraction. ApoE can be synthesized directly within the retina (Muller cells) and binds to receptors on ganglion cells. The subspecies of HDL containing ApoE (HDL-E) supplies lipids and lipid-soluble lutein and zeaxanthin, to the retina. Thus, by increasing HDL levels, retinal lutein and zeaxanthin levels may be concomitantly increased.

Docosahexaenoic Acid (DHA)

Docosahexaenoic acid (DHA) is a key fatty acid found in the retina and is usually present in large amounts in this tissue. DHA intake is inversely related to risk of AMD. A correlation was found between the level of fish intake, a major source of DHA, and AMD risk. Participants who ate fish >4 times/wk had a lower risk of AMD than did those who consumed <3 times/mo (RR:0.65; 95% CI: 0.46, 0.91). These results are consistent with earlier reports in which fish consumption was associated with a decrease risk of advanced AMD. Although DHA's role in retinal function in unknown. Rod outer segments of vertebrate retina have a high DHA content. Since photoreceptor outer segments are constantly being renewed, a constant supply of DHA may be required for proper retinal function and a marginal depletion may impair retinal function and influence the development of AMD. The invention is based, in part, on the observation that supplemental DHA increases HDL and HDL subfractions in humans. In one aspect, the invention provides methods of increasing macular pigment (MP) via an increased transport of lutein into the retina when lutein is taken in conjunction with DHA.

As shown in the Examples, the individual and combined effect of supplemental lutein and DHA on serum and macular concentrations of lutein was studied. To date, the effects of DHA on macular pigmentation have not been explored. The examples also show the effects of DHA supplementation (with or without lutein) on serum lipoproteins and lipoprotein subfractions. The invention demonstrates that changes in lipoproteins, particularly HDL (the major carrier of lutein), caused by DHA supplementation lead to increased changes in macular pigment optical densities when DHA supplementation is combined with lutein supplementation.

The invention pertains to increasing MPOD through the administration of a combination of lutein and DHA. The combination of lutein and DHA has a synergistic effect resulting in increasing MPOD and increasing cognitive function and memory. The content can range from about 0.25 mg to 30 mg of lutein and from about 100 mg to 2 g of DHA, preferably from about 5 mg to 20 mg of lutein and from about 500 mg to 1.5 g of DHA, particular preferably from about 10 mg to 20 mg of lutein and from about 700 mg to 1.5 g of DHA, and more preferably from about 10 to 15 mg of lutein and from about 700 mg to 1000 mg of DHA.

The mixture of lutein and DHA is preferably given in a single dose. In some embodiments lutein and DHA can be taken in separate capsules at the same time. The single dose can be solid, liquid, applied topically or intravenous. In a preferred embodiment, the lutein and DHA are contained in a solid preparation that can be taken orally. In some embodiments, the solid preparation may be combined with a lipophilic component. The utilization of carotenoids, such as lutein, is facilitated when taken in combination with dietary fat. The solid preparation can, for example, use a permissible oil, such as sesame seed oil, corn oil, cotton seed oil, flax seed oil, soybean oil or peanut oil, and esters of medium-chain plant fatty acids at a concentration of from 0 to 500% by weight, preferably from 10 to 300% by weight, particularly preferably from 20 to 100% by weight, based on the active compounds. The solid preparation can also be taken with a meal containing a sufficient fat content (e.g. greater than 1 gram, preferably greater than 10 g, more preferably greater than 25 g) so that the substantially water immiscible carotenoids can be fully absorbed by the subject. Combining the carotenoid preparation with a lipophilic component increases the antioxidant capacity in the aqueous and lipid compartments of plasma.

As shown in the Examples, following supplementation of lutein and DHA, the subjects demonstrated significantly improved results in four cognitive tests, which are have used extensively in cognitive aging research and have been used to demonstrate sensitivity to drugs or other health variables in treatment and epidemiological studies. The results demonstrate that DHA and lutein can act synergistically to improve cognitive function and memory.

Crossing the Blood Brain Barrier

The blood-brain barrier (BBB), while providing effective protection to the brain against circulating toxins, also creates major difficulties in the pharmacological treatment of brain diseases such as memory disorders, Alzheimer's disease, Parkinson's disease, and brain cancer. Most charged molecules, and most molecules over 700 Daltons in size, are unable to pass through the barrier, and smaller molecules may be conjugated in the liver. These factors create major difficulties in the pharmacological treatment of diseases of the brain and central nervous system (CNS). Many therapeutic agents for the treatment of diseases and disorders of the brain and CNS are sufficiently hydrophilic to preclude direct transport across the BBB. Furthermore, these drugs and agents are susceptible to degradation in the blood and peripheral tissues that increase the dose necessary to achieve a therapeutically effective serum concentration.

Brain endothelial cells (BEC) lining cerebral vessels are joined by continuous tight junctions that convert the endothelial cell layer into a highly selective interface separating the peripheral circulation from the brain. The central nervous system (CNS) is dependent on essential lipids that are transported in association with peripheral lipoproteins. Delivery across the blood-brain barrier (BBB) employs specific lipoprotein receptor systems. Patients suffering HDL-deficiency suffer from severe neuropathologies, which illustrates the importance of functional high density lipoprotein (HDL) metabolism for the nervous system. HDL metabolism at the BBB can be used in the delivery of essential metabolites into the brain, protection of BBB-integrity during inflammatory conditions and shuttling of neurotoxic compounds from the brain back into the circulation. In one aspect of this invention, the transport of carotenoids across the blood brain barrier can be increased when taken in combination with a synergistic dose or DHA. The dose of DHA is taken in sufficient quantity to increase HDL and HDL subfractions, thereby improving the delivery of carotenoids into the brain.

Monitoring Treatment

Improved memory and/or cognition can be measured following supplementation through a battery of genitive tests for memory and processing speed, or attention. Examples of such tests include verbal fluency, digit span forward and backward, shopping list task, work list memory test, MIR (memory in reality) test, NES2 pattern comparison test, and the stroop test, which are described further in the Examples section. In addition, commonly used tests to monitor dementia are the Wechsler Adult Intelligence Scale and the Cambridge Cognitive Test (CAMCOG). These tests have a number of different sections and test a variety of things, including the ability to learn new things and the ability to comprehend arithmetic and vocabulary.

Alternatively, regeneration of neurons and hence treatment of disease can be monitored by measuring specific neurotransmitters. For example dopamine levels can be monitored using known methods following administration of the composition of the present invention. To measure dopamine content, a labeled tracer is administered to the subject. The detection of the label is indicative of dopamine activity. Preferably, the labeled tracer is one that can be viewed in vivo in the brain of a whole animal, for example, by positron emission tomograph (PET) scanning or other CNS imaging techniques. See, for example, U.S. Pat. No. 6,309,634 for methods of measuring dopamine content in vivo. By treatment of disease, as used herein, is meant the reduction or elimination of symptoms of the disease of interest, as well as the regeneration of neurons. Thus, dopamine levels prior and subsequent to treatment can be compared as a measure of neuron regeneration.

Pharmaceutical Compositions

The pharmaceutical compositions of the invention can be prepared in various manners well known in the pharmaceutical art. The carrier or excipient may be a solid, semisolid, or liquid material that can serve as a vehicle or medium for the active ingredient. Suitable carriers or excipients are well known in the art and include, but are not limited to saline, buffered saline, dextrose, water, glycerol, ethanol, and combinations thereof. The pharmaceutical compositions may be adapted for oral, inhalation, parenteral, or topical use and may be administered to the patient in the form of tablets, capsules, aerosols, inhalants, solutions, suspensions, powders, syrups, and the like. As used herein, the term “pharmaceutical carrier” may encompass one or more excipients. In preparing formulations of the compounds of the invention, care should be taken to ensure bioavailability of an effective amount of the agent. Suitable pharmaceutical carriers and formulation techniques are found in standard texts, such as Remington's Pharmaceutical Sciences, Mack Publishing Co., Easton, Pa.

Compositions will comprise a sufficient combination of DHA and at least one carotenoid, such as lutein, to produce a therapeutically effective amount, i.e., an amount sufficient to reduce or ameliorate symptoms of the disease state in question or an amount sufficient to confer the desired benefit. The compositions can contain a pharmaceutically acceptable carrier. Such carriers include any pharmaceutical agent that does not itself induce the production of antibodies harmful to the individual receiving the composition, and which may be administered without undue toxicity. Pharmaceutically acceptable carriers include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable carriers and excipients is available in Remington's Pharmaceutical Sciences (Mack Pub. Co., N.J. 1991).

For oral administration, the compounds can be formulated into solid or liquid preparations, with or without inert diluents or edible carrier(s), such as capsules, pills, tablets, troches, powders, solutions, suspensions or emulsions. The tablets, pills, capsules, troches and the like also may contain one or more of the following adjuvants: binders such as microcrystalline cellulose, gum tragacanth or gelatin; excipients such as starch or lactose; disintegrating agents such as alsinic acid, Primogel™, corn starch and the like; lubricants such as stearic acid, magnesium stearate or Sterotex™; glidants such as colloidal silicon dioxide; sweetening agents such as sucrose or saccharin; and flavoring agents such as peppermint, methyl salicylate or fruit flavoring. When the dosage unit form is a capsule, it also may contain a liquid carrier such as polyethylene glycol or fatty oil. Materials used should be pharmaceutically pure and non-toxic in the amounts use. These preparations should contain at least 0.05% by weight of the therapeutic agent, but may be varied depending upon the particular form and may conveniently be between 0.05% to about 90% of the weight of the unit. The amount of the therapeutic agent present in compositions is such that a unit dosage form suitable for administration will be obtained.

The solutions or suspensions also may include one or more of the following adjuvants depending on the solubility and other properties of the therapeutic agent: sterile diluents such as water for injections, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates; and agents for the adjustment of toxicity such as sodium chloride or dextrose.

The exact amount of a therapeutic of the invention that will be effective in the treatment of a particular disease or disorder will depend on a number of factors that can be readily determined by the attending diagnostician, as one of ordinarily skilled in the art, by the use of conventional techniques and by observing results obtained under analogous circumstances. Factors significant in determining the dose include: the dose; the species, subject's size, age and general health; the specific disease involved, the degree of or involvement of the severity of the disease; the response of the individual patient; the particular compound administered; the mode of administration; the bioavailability characteristics of the preparation administered; the dose regimen selected; the use of concomitant medication; and other relevant circumstances specific to the subject. Effective doses optionally may be extrapolated from dose-response curves derived from in vitro or animal model test systems. In general terms, an effective amount of the combination of DHA and lutein of the instant invention to be administered systemically on a daily basis is about 10-30 mg/kg DHA and 0.08-0.5 mg/kg lutein.

In certain embodiments, the composition of the invention may be orally administered, for example, with an inert diluent or an assimilable edible carrier. The composition (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like.

It is also possible to use dry powders that comprise the inventive carotenoid and DHA combinations to enrich milk products such as yogurt, flavored milk drinks or ice cream, or milk pudding powders, baking mixes and confectionery products, for example fruit gums.

The invention also relates to food supplements, animal feeds, foods and pharmaceutical and cosmetic preparations comprising the above-described preparations, in particular carotenoid formulations of mixtures lutein and DHA. Food supplement preparations and pharmaceutical preparations that comprise the inventive dry powders include, but are not limited to, tablets, sugar-coated tablets and hard and soft gelatin capsules. Preferred food supplement preparations are tablets into which the dry powders are co-incorporated, and soft gelatin capsules in which the carotenoid-containing multicore structures are present as oily suspension in the capsules. The carotenoid content in these capsules is from 0.1 to 30 mg of lutein and 500 mg to 2 g of DHA, preferably from about 6 to 15 mg of lutein and from 700 mg to 1.5 g of DHA.

In certain embodiments, the composition of the present invention can be administered in a liquid form. The pharmacological agent of the present invention is freely soluble in a variety of solvents, such as for example, methanol, ethanol, and isopropanol. The pharmacological agent is, however, highly lipophilic and, therefore, substantially insoluble in water. A variety of methods are known in the art to improve the solubility of the pharmacological agent in water and other aqueous solutions. For example, U.S. Pat. No. 6,008,192 to Al-Razzak et al. teaches a hydrophilic binary system comprising a hydrophilic phase and a surfactant, or mixture of surfactants, for improving the administration of lipophilic compounds such as the pharmacological agent of the present invention.

Supplementary active compounds can also be incorporated into the compositions. In some embodiments, the composition of the invention can be coformulated with and/or coadministered with one or more additional carotenoid or antioxidant. For example, the composition can include a combination of DHA and lutein, together with Alpha-carotene, β-carotene, lycopene, β-cryptoxanthin, and zeaxanthin. In certain embodiments, the composition of the invention is coformulated with and/or coadministered with one or more additional therapeutic agents that are useful for improving the pharmacokinetics of the pharmacological agent. A variety of methods are known in the art to improve the pharmacokinetics of the pharmacological agent of the present invention. For example, U.S. Pat. No. 6,037,157 to Norbeck et al. discloses a method for improving the pharmacokinetics of the pharmacological agent by coadministration of the pharmacological agent and a drug that is metabolized by the cytochrome P450 monooxygenase, such as for example, the P450 3A4 isozyme.

The composition of the present invention can be used alone or in combination to treat neurodegenerative disorders to produce a synergistic effect. Likewise, the pharmacological agent can be used alone or in combination with an additional agent, e.g., an agent which imparts a beneficial attribute to the therapeutic composition, e.g., an agent which effects the viscosity of the composition. The combination can also include more than one additional agent, e.g., two or three additional agents if the combination is such that the formed composition can perform its intended function.

The pharmaceutical compositions of the invention may include a “therapeutically effective amount” or a “prophylactically effective amount” of a pharmacological agent of the invention. A “therapeutically effective amount” refers to an amount effective, at dosages and for periods of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of the composition of the invention is between 10-30 mg/kg body weight DHA and 0.08-0.5 mg/kg body weight of lutein. Preferably, administration of a therapeutically effective amount of DHA and lutein in a concentration of pharmacological agent in the bloodstream that is between about 30-1500 μM DHA and 0.1-250 μM lutein. More preferably, the concentration of pharmacological agent in the blood is between about 100-1000 μM DHA and 1-10 μM lutein. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Uses

The methods of the present invention can be used to slow or prevent neurodegeneration. In addition, the methods of the invention can be used to prevent and/or slow the progression of macular degeneration. Many of these diseases are associated with the normal aging process, and thus the supplement of the present invention can be take prophylatically to slow progression of the disease.

In addition, many disorders or diseases which arise due to oxidative stress and the presence of free radicals can be improved using the methods of the present invention. The methods of the present invention can be used to reduce, ameliorate, prevent, and/or treat disorders associated with antioxidant levels and excess free radicals. Populations at risk can be identified through methods known in the art (See, for example, U.S. Publication No. US 2002-0182736 A1, U.S. patent application Ser. No. 10/114,181 filed Apr. 2, 2002, which describes a method that is accurate, quick, non-invasive, which can be easily adapted for high throughput usage and diagnostic procedures). At risk populations or people who wish to reduce the risk of free-radical associated disorders can benefit from the methods of the present invention. For example, disorders that can be reduced, ameliorated, prevented, and/or treated using the methods of this invention include, but are not limited to, aging at a higher than normal rate, segmental progeria disorders, Down's syndrome; heart and cardiovascular diseases such as arteriosclerosis, adriamycin cardiotoxicity, alcohol cardiomyopathy; gastrointestinal tract disorders such as inflammatory & immune injury, diabetes, pancreatitis, halogenated hydrocarbon liver injury; eye disorders such as cataractogenesis, degenerative retinal damage, macular degeneration; kidney disorders such as autoimmune nephrotic syndromes and heavy metal nephrotoxicity; skin disorders such as solar radiation, thermal injury, porphyria: nervous system disorders such as hyperbaric oxygen, Parkinson's disease, neuronal ceroid lipofuscinoses, Alzheimer's disease, muscular dystrophy and multiple sclerosis; lung disorders such as lung cancer, oxidant pollutants (O₃,NO₂), emphysema, bronchopulmonary dysphasia, asbestos carcinogenicity; red blood cell disorder such as malaria Sickle cell anemia, Fanconi's anemia and hemolytic anemia of prematurity; iron overload disorders such as idiopathic hemochromatosis, dietary iron overload and thalassemia; inflammatory-immune injury, for example, glomerulonephritis, autoimmune diseases, rheumatoid arthritis; ischemia reflow states disorders such as stroke and myocardial infarction; liver disorder such as alcohol-induced pathology and alcohol-induced iron overload injury; and other oxidative stress disorders such as AIDS, radiation-induced injuries (accidental and radiotherapy), general low-grade inflammatory disorders, organ transplantation, inflamed rheumatoid joints and arrhythmias.

This invention is further illustrated by the following examples, which should not be construed as limiting. The contents of all references, patents and published patent applications cited throughout this application, are incorporated herein by reference.

### EXAMPLES

A study was undertaken to evaluate the effectiveness of the composition of the present invention and its effect on the patients. In a randomized double-blind study on the effect of supplemental lutein and docosahexaenoic acid (DHA) in comparison to placebo on visual function, the 50 female subjects, aged 60 to 80 years, also completed cognitive tests measuring verbal fluency, memory, processing speed and accuracy, and self-reports of mood. After supplementation, subjects in DHA (n=14, p=0.03), lutein (n=11, p=0.000), or DHA and lutein (n=14, p=0.000) intervention groups increased the number of items within a category they could name within one minute. Subjects receiving DHA and lutein learned all items on a shopping list more quickly (p=0.03) and recalled more common household items after a delay (p=0.02). There was also a trend suggesting more efficient learning by subjects receiving DHA and lutein in recalling lists of random words (p=0.07). Increases in speed and accuracy and improvement in reports of mood were not found in the supplemented groups, although subjects in the placebo (n=10) group increased their speed (p=0.04) but not their accuracy in choosing the odd visual display in a test of choice response time. These results, discussed below, indicate that DHA and lutein have a synergistic effect in improving cognition and memory. The oral intake of the composition can be used either therapeutically or prophylactically to improve memory of a subject and reduce dementia.

### Example 1

### Study Design and Methods

Subjects: Fifty non-smoking women (60-80 years) were recruited from the general population for a 4 month supplementation study. All subjects underwent a screening examination that includes a medical history, a physical examination, and a routine blood clinical chemistry profile. Volunteers with any history or biochemical evidence of lactose intolerance, liver, kidney, or pancreatic disease, anemia, active bowel disease or resection, insulin-dependent diabetes, easy bruising or bleeding, bleeding disorders, hyperglyceridemia, hyperlipidproteinemia, or alcoholism were excluded from the study. Moreover, individuals taking mineral oil or medications suspected of interfering with fat-soluble vitamin absorption were excluded. Subjects using steroids or non-steroid anti-inflammatory drugs, or antihistamine drugs were excluded. Subject who had a vaccination within 2 weeks of the study screening were be excluded. Subjects were excluded if they have taken any nutrient supplement for 2 months or more before admission into the study or carotene supplements 6 months or more before the study. Smoking was not permitted during the course of the study.

All subjects were given a complete ophthalmic examination including fundus photography before and after their participation in the study. During this procedure, eyedrops (Phenylephrine 2.5% and Tropicamide 1%) were used to dilate the eyes. These drops are used by most ophthalmologists. The intraocular pressure was measured with a device that makes direct contact with the cornea and a local anaesthetic was applied to both eyes. This is standard clinical procedure. Eye diseases such as macular degeneration, glaucoma, cataract or cataract surgery were exclusions for participation in the study. This study protocol was approved by the Human Investigative Review Committee of Tufts University, Tufts-New England Medical Center and the Schepens Eye Research Institute. Informed consent was obtained from all subjects.

Study Design.

Women were randomly assigned to one of four groups: Control (n=10), DHA (n=14, DHA 800 mg/d), lutein (n=12, lutein 12 mg/d), and lutein+DHA (n=14, lutein 12 mg/d; DHA 800 mg/d) (C, D, L, and LD, respectively). The supplement type was randomized. Subjects visited the Jean Mayer USDA Human Nutrition Research Center on Aging at Tufts University on days that supplements were distributed and blood obtained (0 mo-baseline, 2 mo, and 4 mo). Subjects were instructed to take the supplement with a nutritional energy drink (8 oz., Boost®, Mead Johnson Nutritionals) but were otherwise asked to not alter their diets. The diet of each subject was monitored with food frequency questionnaires (Block G, et al., A data-based approach to diet questionnaire design and testing. Am J Epidemiol, 1986. 24: p. 453-469) completed at baseline, 2 months and 4 months to be sure that there were no confounding changes in dietary intake. All subjects completed at least two sets of cognitive and mood self-report tests, at the beginning of the study, then at the end of the study, after four months of supplementation, at the Schepens Eye Research Institute. Compliance was by monitored by interview, compliance calendars and capsule count. Both the subjects and the experimenter were masked to the experimental groups. Blood samples were collected at baseline, 2 months and 4 months, and serum was separated from red blood cells (800×g, 10 minutes). Aliquots of serum are stored at −70° C. until analyzed. At baseline and 2 months, a two month supply of placebo supplements, DHA (800 mg/d, DHASCO, Martek Biological Sciences), lutein (12 mg/d plus ˜0.5 mg zeaxanthin, Kemin Foods), or lutein+DHA (12 mg/d and 800 mg/d, respectively and nutritional energy drink was provided. At baseline and 4 months, subjects visited the Schepens Eye Research Institute (Boston, Mass.) for testing of MP optical density (MPOD) and an ophthalmic exam. There were two visits at each time point.

Methods:

Supplementation Protocol. Dietary supplements in capsule form were Lutein (12 mg/day) (Kemin Foods) and Docosahexaenoic Acid (DHASCO) (800 mg/day) (Martek Biological Sciences). Participants in each of the four supplementation groups were instructed to drink one can of a nutritional supplement daily (Boost Plus) (Mead Johnson) when taking their capsules. Subjects ingested their dietary supplements daily for a period of 4 months. Both the subjects and the experimenter were masked to the experimental groups.

Tests and Measurements. The battery of cognitive tests included tests of memory and processing speed or attention and a measure of self-reported mood. All of these tests or versions of them have been used in cognitive aging research and also have demonstrated sensitivity to drugs or other health variables in treatment or epidemiological studies (See, for example, Ferris, S. H., et al. (1986). Assessing cognitive impairment and evaluating treatment effects: Psychometric performance tests. In L. Poon (Ed.), Handbook for Clinical Memory Assessment of Older Adults (pp. 139-148). Washington, D.C.: American Psychological Association; Letz, R. (1991). NES2 User's Manual (Version 4.4), Winchester, Mass.: Neurobehavioral Systems, Inc.; Johansson, B. & Zarit, S. H. (1997). Early cognitive markers of the incidence of dementia and mortality: A longitudinal population-based study of the oldest old. International Journal of Geriatric Psychiatry, 12, 53-59; Payton, M., Riggs, K. M., Spiro III, A., Weiss, S. T. & Hu, H. (1998). Relations of bone and blood lead to cognitive function: The VA Normative Aging Study. Neurotoxicology and Teratology, 20, 1-9). Alternate forms of Verbal Fluency and memory tests were administered at test sessions in order to decrease practice effects.

Verbal Fluency Test: Subjects name as many items from a category as possible during a one-minute period. (See, for example, Borkowski, J. G., Benton, A. L., & Spreen, O. (1967). Word fluency and brain damage. Neuropsychologia, 5, 135-150).

Digit Span Forward and Backward: Subjects are asked to repeat numbers in increasing spans in forward sequences, then in backward sequences. Protocol adapted from Wechsler, D. A. (1981). Manual for the Wechsler Adult Intelligence Scale—Revised. New York: Psychological Corporation.

Shopping List Task: Ten associated words (common food items found in a supermarket) are read to the subject in up to five verbally presented serial trials. Verbal recall is tested immediately after each trial and after a delay (McCarthy, M., et al. (1981). Acquisition and retention of categorized material in normal aging and senile dementia. Experimental Aging Research, 7 127-135.)

Word List Memory Test: Ten unassociated words are presented (at a rate of one word every two seconds) on a computer monitor in three serial trials. Verbal recall is tested immediately after each trial and after a delay. (Computer version of test described by Morris, J. C., et al. (1989). The consortium to establish a registry for Alzheimer's disease (CERAD). Part I. Clinical and neuropsychological assessment of Alzheimer's disease. Neurology, 39, 1159-1165.)

MIR (Memory in Reality) Test: Subjects place 10 common household objects in 7 rooms of a model of an apartment. Verbal and visuospatial (location) recall is tested after a delay. (For protocol, see Johansson, B. (1988/89). The MIR—Memory-in-Reality Test. Psykologiforlaget AB, Stockholm.)

NES2 Pattern Comparison Test: Subjects choose the odd pattern from three similar patterns displayed on a computer monitor. The scores are the number of correct responses (maximum 15) and the mean response latency for correct decisions. (See Letz, R. (1991). NES2 User's Manual (Version 4.4), Winchester, Mass.: Neurobehavioral Systems, Inc.)

Stroop Test: Subjects name words (subtask 1—read words printed in black, and subtask 2—read color name words printed in the same color) and colors (subtask 3—name colors of rectangles, and subtask 4—name colors in which color name words are printed, when colors are different from the color name) in this assessment of response time and the ability to inhibit non-salient information. This version is presented via computer. Protocol adapted from Stroop, J. R. (1935). Studies of interference in serial verbal reactions. Journal of Experimental Psychology, 18, 643-662.

NES2 Mood Scales: Subjects rate their degree of tension, depression, anger, fatigue, and confusion over the previous seven days, using a computerized format. The Mood Scales are adapted from the Profile of Mood States (POMS; McNair, Lorr, & Dropleman, 1971). For description, see Letz, R. (1991). NES2 User's Manual (Version 4.4), Winchester, Mass.: Neurobehavioral Systems, Inc.

Serum Analysis for Carotenoids and Fatty Acid:

Serum carotenoids were extracted and analyzed by HPLC using the method described by Yeum et al. (Yeum, K. J., et al., Am J Clin Nutr, 1996. 64(4): p. 594-602.). The fatty acid composition of serum was determined following direct transesterification and gas-liquid chromatography by procedures similar to those previously described (Patton, G. M., et al., Methods in Enzymology, 1981. 72: p. 8-20.).

Measurement of Macular Pigment Optical Density (MPOD):

The method of assessing MPOD involves a three-channel Maxwellian view optical system and used a psycho physical method described by Hammond et al. (Hammond B. R., et al., Vision Res, 1996.36: p. 2001-2012). A rotating sectored mirror combined two channels to produce the test stimulus, which alternated between a measuring and a reference field. The third channel provided a 10 degree background field. Ditric Optics interference filters were used to set the wavelength of the background (470 nm) and reference (550 nm) channels. A grating monochromator (Bausch and Long, Model, No. HD426) was used to determine the wavelength of the measuring (460 nm) stimulus. The stimulus subtended 0.8 degrees of visual angle and was centrally fixated for measurements of peak MP density Additional measurements of macular pigment were obtained by having the subject look at a fixation point at 1.5°, 3° and 5° temporal retinal eccentricities. The fixation point was produced by a small black dot on transparent glass in the path of the light that formed the background field. The parafoveal reference was located at 7° temporal retinal eccentricity. The subject's head position was stabilized with an adjustable bitebar and headrest apparatus. Thus, a profile of MPOD in the temporal retina was obtained for each subject. Measurements were made in the right eye for all subjects. MPOD was measured in two baseline sessions on separate days and in two sessions on separate days at the end of 4 months of supplementation.

Lipoprotein Analysis:

At study end (4 mo), serum was analyzed for total cholesterol, lipoproteins (VLDL, LDL, HDL) as well as lipoprotein subfractions using NMR spectroscopy (Otvos, J. D., Clin Lab, 2002. 48: p. 171-180). The NMR method employs the characteristic methyl group signals broadcast by lipoprotein subclasses of different size as the basis for their quantification. Each measurement includes the concentrations of 5 subclasses of HDL (larger numbers denoting larger subclasses), 3 subclasses of LDL and 6 subclasses of VLDL (V1-V6). Lipoprotein subclasses were grouped into large, intermediate and small subclasses. That is, large, intermediate, and small HDL were H5+H4, H3, and H2+H1, respectively. Large, intermediate and small LDL were L3, L2, and L1, respectively. And large, intermediate and small VLDL were V6+V5, V4+V3, and V2+V1, respectively.

Statistical Analyses:

Results are expressed as mean±SE. Within each group, each subject was followed longitudinally and significant differences from baseline were measured using Student's paired t-test (Systat version 10, Port Richmond, Calif. Group differences were measured using ANOVA followed by the Bonferroni post hoc test (Systat 10).

Differences between cognitive and mood scores at baseline and after supplementation were tested by Student's t test for each treatment group. In the case of those variables where a significant change was found from baseline to end of study, correlations were calculated between age, education, serum levels of DHA and lutein, and test scores, for those test scores where the distribution was normal or near-normal. Regression analyses were used to further examine significant associations (p<0.05) or those marginally significantly (p<=0.10) in these initial analyses. In those cases in which age or education were significantly related to performance on a particular test, they were entered as covariates. Statistical analyses were carried out using Systat version 9. The total MPOD was calculated as the area under the curve for the 5 loci at which optical densities were measured KaleidaGraph version 3.5, Synergy Software, Reading, Pa.). Within each group, each subject was followed longitudinally and significant differences from baseline were measured using Wilcoxan signed rank test. Group differences were measured using Kruskal Wallis analysis of variance followed by Mann-Whitney U test statistical analysis using SAS version 8 (SAS version 8, cary, NC, SAS Institute, Inc, 1999). Significance was considered when the p-value was less than 0.05.

### Example 2

### Results

Subjects.

Fifty-seven women were admitted for this study. Seven women dropped out of the study for the following reasons: medication use (1), autoimmune disease (1), unknown (1), aversion to study protocol (4). Therefore, the total number of women studies was 50. Subject characteristics at baseline are given in Table 1. Table 1 presents the age and education characteristics of each of the four study groups. At baseline, neither age nor years of education in the total sample (n=49) was significantly associated with cognitive test scores or self-reported mood scores. There were no significant differences in baseline measures of age, body mass index (kg/m²), serum concentrations of lutein and DHA, or MPOD. One subject in the LD group had an increase in the confluence of soft drusen. No other subjects had indications of ocular changes. In this age group it is not surprising that an occasional subject might show changes with time that are unrelated to the supplementation. Compliance to consuming the nutrient drink and supplements (days supplements consumed/total days of study) was 97%. Furthermore, there was not a significant change in body weight within any of the four groups at study end. Dietary intakes of lutein and DHA were not different among groups and did not change during the study. Dietary intakes of lutein were 3-7 times lower than the study intervention of 12 mg/d and dietary intakes of DHA were 3-16 times lower than the intervention of 800 mg/d.

TABLE 1
Age and Years of Education of Treatment Groups

| | Placebo | DHA | Lutein | DHA and Lutein |
|---|---|---|---|---|
| | (N = 10) | (N = 14) | (N = 12) | (N = 14) |
| Age (mean ± se). | 68 ± 1 | 68 ± 1 | 65 ± 2 | 68 ± 1 |
| (range) | 62-73 | 60-77 | 60-77 | 61-80 |
| Body mass index, | 23.8 ± 3.1 | 24.5 ± 1.3 | 24.6 ± 1.5 | 27.0 ± 1.3 |
| kg/m² | | | | |
| Education (Years) | 13.6 (3.5) | 16.0 (3.6) | 13.8 (1.8) | 14.8 (2.0) |
| (range) | 6-18 | 12-25 | 11-16 | 12-18 |
| Serum lutein | 0.30 ± 0.05 | 0.37 ± 0.04 | 0.28 ± 0.04 | 0.32 ± 0.03 |
| μmol/L) | | | | |
| Serum DHA | 31.7 ± 3.3 | 26.2 ± 2.1 | 36.5 ± 2.1 | 30.1 ± 3.7 |
| (nmol/L) | | | | |
| MP density | 1.05 ± 0.14 | 0.92 ± 0.09 | 1.09 ± 0.10 | 1.01 ± 0.13 |
| (optical density) | | | | |

Table 2 presents the means and standard deviations of test scores by subject group at baseline and after supplementation. The average performance of subjects was close to ceiling (the maximum score) for many cognitive tests, which suggests that older and less educated subjects in this study were generally very competent.

TABLE 2
Means (Standard Deviations) of Scores at Baseline and after Supplementation.

| | Placebo | | DHA | |
|---|---|---|---|---|
| Measure | Baseline | Final | Baseline | Final |
| Verbal Fluency | 12.9 (6.2) | 13.8 (3.5) | 15.0 (4.9) | 17.8 (3.1)** |
| Forward Digit Span | | | | |
| Forward Digit Span Length | 7.2 (1.2) | 7.2 (1.4) | 6.6 (1.5) | 6.7 (1.3) |
| Forward Digit Span Total | 9.7 (2.5) | 9.0 (2.4) | 8.4 (2.8) | 8.5 (2.7) |
| Backward Digit Span | | | | |
| Backward Digit Span Length | 5.9 (1.4) | 5.8 (1.7) | 5.4 (1.6) | 5.8 (1.6) |
| Backward Digit Span Total | 8.2 (2.7) | 8.4 (3.3) | 7.9 (3.1) | 8.4 (3.2) |
| Shopping List Memory Test | | | | |
| Trial 1 Items Recalled (max. 10) | 6.5 (1.2) | 7.7 (1.5) | 7.2 (1.4) | 7.7 (1.7) |
| Trials to Learned List (max. 6) | 3.0 (0.8) | 2.8 (0.9) | 3.1 (1.3) | 2.6 (1.3) |
| Delayed Recall (max. 10) | 9.5 (0.9) | 9.5 (0.7) | 9.0 (0.9) | 8.7 (1.7) |
| Word List Memory Test | | | | |
| Trial 1 Items Recalled (max. 10) | 6.2 (1.3) | 6.6 (1.8) | 6.3 (1.7) | 5.9 (1.5) |
| Trials to Learned List (max. 4) | 3.1 (0.9) | 2.8 (0.9) | 3.0 (1.0) | 3.0 (0.7) |
| Delayed Recall (max. 10) | 8.1 (1.1) | 8.3 (1.8) | 8.1 (1.1) | 8.6 (1.3) |
| MIR Apartment Test | | | | |
| Delayed Recall (max. 10) | 9.3 (0.8) | 9.4 (0.7) | 9.4 (0.9) | 9.4 (0.8) |
| Location Recall (max. 10) | 9.7 (0.7) | 9.7 (0.7) | 9.9 (0.3) | 10.0 (0) |
| Pattern Recognition Test | | | | |
| Number Correct (max. 15) | 14.5 (0.7) | 14.9 (0.3) | 14.6 (0.9) | 14.6 (0.5) |
| Mean Response Time-Correct (s) | 6.8 (3.0) | 5.9 (2.3)* | 5.4 (2.1) | 5.0 (0.7) |
| Stroop Test | | | | |
| Mean RT, Read Words-Black (ms) | 1040 (380) | 891 (222) | 879 (429) | 748 (157) |
| Mean RT, Read Words-Color (ms) | 788 (200) | 804 (202) | 715 (181) | 727 (132) |
| Mean RT, Name Colors (ms) | 919 (173) | 951 (220) | 838 (161) | 884 (163) |
| Mean RT, Name Colors-Words(ms) | 1419 (308) | 1413 (508) | 1269 (215) | 1277 (226) |
| Total RT, Interference (NC − C)(s) | 25.0 (14.8) | 23.1 (22.0) | 21.5 (10.0) | 19.7 (8.3) |
| Mood Scales | | | | |
| Tension | 2.3 (0.9) | 2.2 (0.8) | 2.0 (0.8) | 2.1 (0.5) |
| Depression | 1.7 (0.7) | 1.9 (0.7) | 1.7 (0.7) | 1.7 (0.7) |
| Anger | 1.7 (0.5) | 1.5 (0.6) | 1.6 (0.7) | 1.6 (0.9) |
| Fatigue | 2.0 (0.7) | 2.1 (0.5) | 2.1 (0.8) | 2.1 (0.7) |
| Confusion | 1.4 (0.2) | 1.7 (0.5) | 1.7 (0.5) | 1.8 (0.5) |
| | Lutein | | DHA and Lutein | |
| Measure | Baseline | Final | Baseline | Final |
| Verbal Fluency | 11.3 (5.1) | 15.5 (5.5)** | 12.1 (2.8) | 16.9 (3.4)** |
| Forward Digit Span | | | | |
| Forward Digit Span Length | 6.6 (1.2) | 7.0 (1.5) | 7.3 (1.3) | 7.3 (1.3) |
| Forward Digit Span Total | 8.1 (2.3) | 8.7 (2.5) | 9.5 (2.5) | 9.6 (2.7) |
| Backward Digit Span | | | | |
| Backward Digit Span Length | 5.1 (1.6) | 4.7 (1.4) | 5.4 (1.4) | 5.9 (1.5) |
| Backward Digit Span Total | 7.5 (3.1) | 6.9 (2.7) | 7.4 (2.6) | 8.4 (2.6) |
| Shopping List Memory Test | | | | |
| Trial 1 Items Recalled (max. 10) | 6.9 (1.8) | 6.5 (2.1) | 7.0 (1.4) | 6.9 (1.6) |
| Trials to Learned List (max. 6) | 4.2 (1.5) | 3.9 (1.4) | 3.9 (1.4) | 2.9 (1.3)** |
| Delayed Recall (max. 10) | 8.3 (1.9) | 7.6 (3.0) | 8.6 (0.6) | 8.9 (1.4) |
| Word List Memory Test | | | | |
| Trial 1 Items Recalled (max. 10) | 5.8 (1.8) | 5.8 (1.8) | 5.6 (1.5) | 6.2 (1.4) |
| Trials to Learned List (max. 4) | 3.4 (0.7) | 3.5 (0.8) | 3.6 (0.6) | 3.0 (0.9)* |
| Delayed Recall (max. 10) | 6.8 (2.9) | 7.6 (2.4) | 7.6 (1.6) | 8.1 (2.0) |
| MIR Apartment Test | | | | |
| Delayed Recall (max. 10) | 8.3 (1.6) | 8.6 (2.1) | 8.3 (1.5) | 9.1 (1.2)** |
| Location Recall (max. 10) | 9.5 (1.0) | 9.5 (0.8) | 9.1 (0.9) | 9.4 (1.2) |
| Pattern Recognition Test | | | | |
| Number Correct (max. 15) | 14.5 (0.9) | 14.3 (1.8) | 14.7 (0.8) | 14.0 (1.2) |
| Mean Response Time-Correct (s) | 6.1 (2.3) | 6.4 (2.3) | 5.9 (1.5) | 5.9 (1.1) |
| Stroop Test | | | | |
| Mean RT, Read Words-Black (ms) | 844 (239) | 945 (185) | 861 (169) | 819 (165) |
| Mean RT, Read Words-Color (ms) | 753 (210) | 883 (213) | 754 (176) | 743 (169) |
| Mean RT, Name Colors (ms) | 1008 (217) | 1014 (193) | 947 (150) | 965 (182) |
| Mean RT, Name Colors-Words(ms) | 1492 (329) | 1462 (221) | 1366 (225) | 1317 (241) |
| Total RT, Interference (NC − C)(s) | 24.2 (10.9) | 22.4 (7.1) | 21.0 (7.8) | 17.6 (8.6) |
| Mood Scales | | | | |
| Tension | 2.1 (0.4) | 2.4 (0.9) | 2.0 (0.6) | 1.9 (0.5) |
| Depression | 1.5 (0.3) | 1.8 (0.7) | 1.7 (0.6) | 1.6 (0.4) |
| Anger | 1.4 (0.4) | 1.5 (0.5) | 1.4 (0.5) | 1.5 (0.4) |
| Fatigue | 2.4 (0.6) | 2.9 (0.9) | 2.3 (0.8) | 2.1 (0.6) |
| Confusion | 1.9 (0.5) | 2.4 (0.9) | 1.9 (0.7) | 1.7 (0.4) |

*p <= 0.10
**p < 0.05

Verbal Fluency:

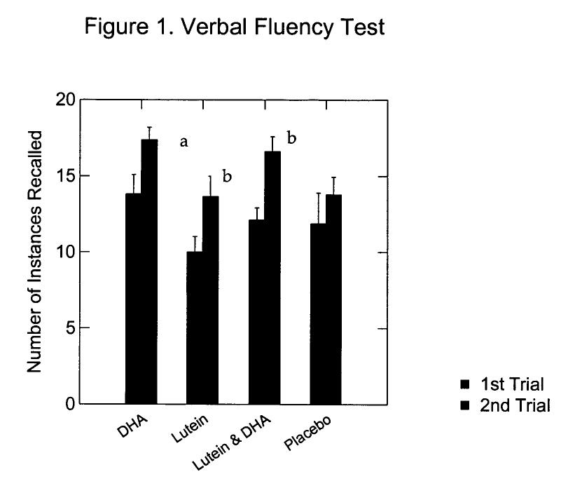

After supplementation, subjects in the DHA (t=−2.43, p=0.03), Lutein (t=−5.61, p=0.000), and DHA and Lutein (t=−5.45, p=0.000) supplement groups named significantly more items from a category within a minute. Control subjects did not name significantly more items. FIG. 1 shows the relative performance of the subject groups.

Memory and Rate of Learning:

None of the subject groups significantly increased the number of items (either length of span or total number of items) they recalled on the short-term memory Forward Digit Span or Backward Digit Span tasks. On the Shopping List and Word List memory tests, none of the subject groups significantly increased the number of items they recalled on the first trial during the study.

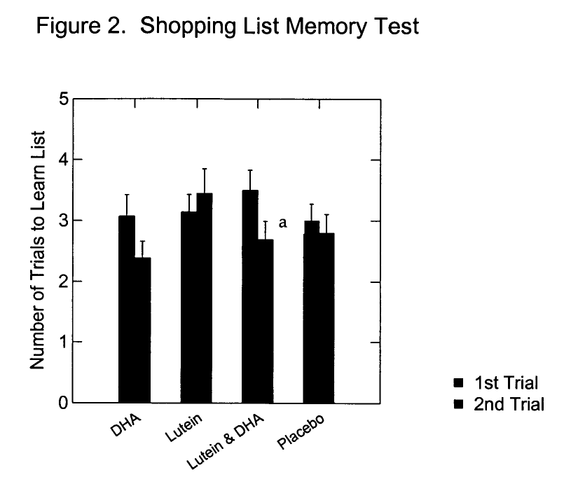

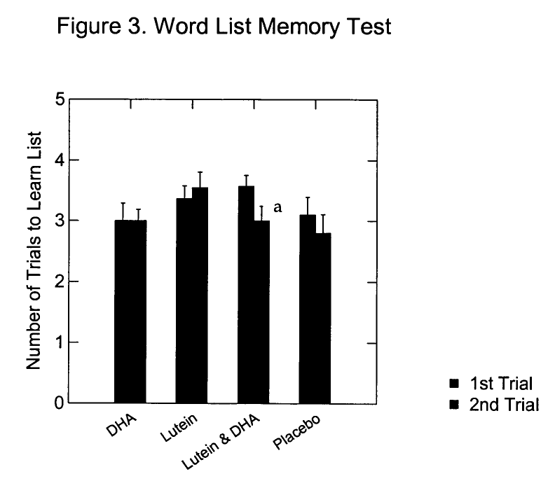

However, on the Shopping List memory test, subjects in the DHA and Lutein (t=2.51, p=0.03) supplement group learned all 10 items significantly faster, within five trials or less, after supplementation (FIG. 2). There was also a trend toward more efficient learning on the Word List memory test, which only had a maximum of three trials in which to learn the list, after DHA and Lutein (t=1.96, p=0.07) supplementation (FIG. 3).

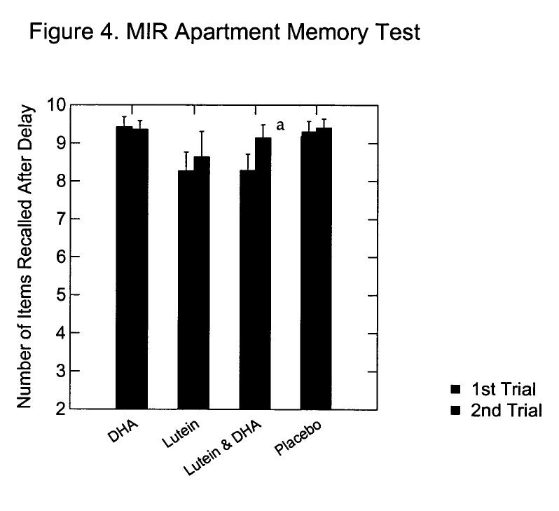

After a delay, the subjects in the DHA and Lutein (t=−2.75, p=0.02) supplement group recalled significantly more items on the MIR Apartment memory test, after supplementation (FIG. 4). None of the subject groups significantly increased the number of items they recalled after a delay on the Shopping List and Word List memory tests.

Speed and Accuracy:

On the Pattern Recognition task, only the subjects in the Placebo group (t=2.38, p=0.04), who originally had the longest response times on average of all the groups, significantly increased their mean response speed for correct decisions. None of the groups increased their accuracy rate significantly (on average, subjects in all groups were close to ceiling). None of the supplementation groups significantly increased their mean response speed for correct decisions.

On the computerized version of the Stroop Test, none of the subject groups significantly changed mean response times for reading words or naming colors on any of the four lists. As a measure of interference, for each subject, total time to name colors of rectangles (subtask 3) was subtracted from the total time to name colors of color name words that were printed in different colors (subtask 4). None of the groups significantly changed on this interference measure either from baseline to end of study.

Mood:

None of the subject groups reported significantly different moods after supplementation.

Serum Lutein:

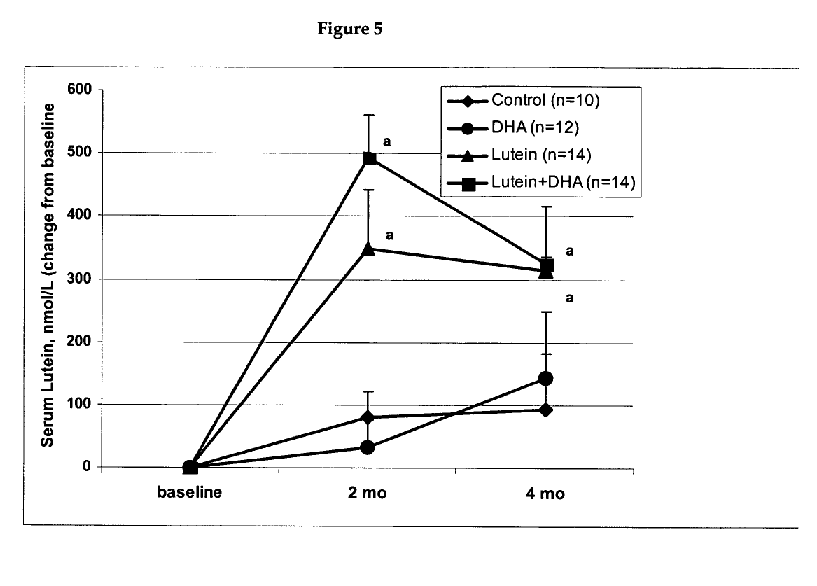

Serum concentrations of lutein significantly increased from baseline after 2 and 4 months of lutein supplementation (with and without DHA) (p<0.05, FIG. 5). No significant changes in serum lutein were observed for the C and D groups. After two months of supplementation the LD group had significantly greater changes in serum concentrations of lutein than the C and D groups (p<0.0002). At study end, changes in serum lutein in the L and LD groups were significantly greater than that in the C and D groups (p<0.0086) but were not different from each other. For all groups, serum concentrations of zeaxanthin did not change throughout the study.

Serum DHA.

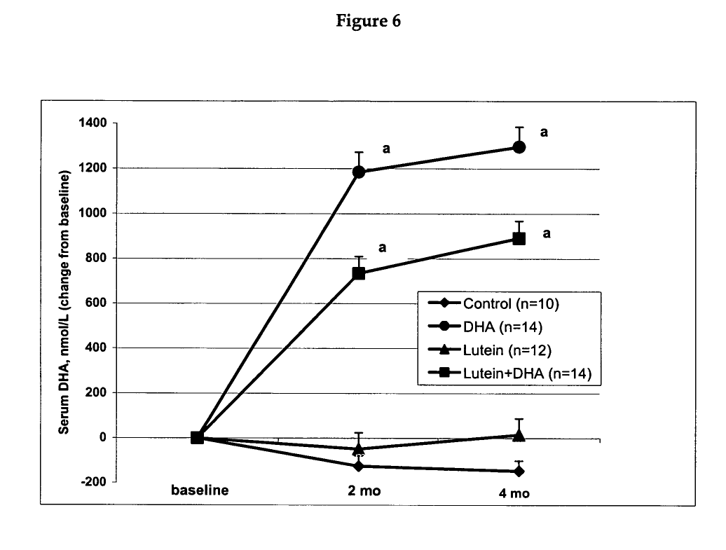

Serum concentrations of DHA significantly increased from baseline after 2 and 4 months of DHA supplementation (with and without lutein) (p<0.001, FIG. 6). No significant changes in serum DHA were observed for the C and L groups except for a significant decrease in the C group at 4 months ((p<0.05). After 2 and 4 months of supplementation the D and LD groups had significantly higher serum concentrations of DHA than the C and L groups (p<0.0001) and the D group had significantly greater serum DHA concentrations that the LD group p<0.05).

Macular Pigment Optical Density (MPOD).

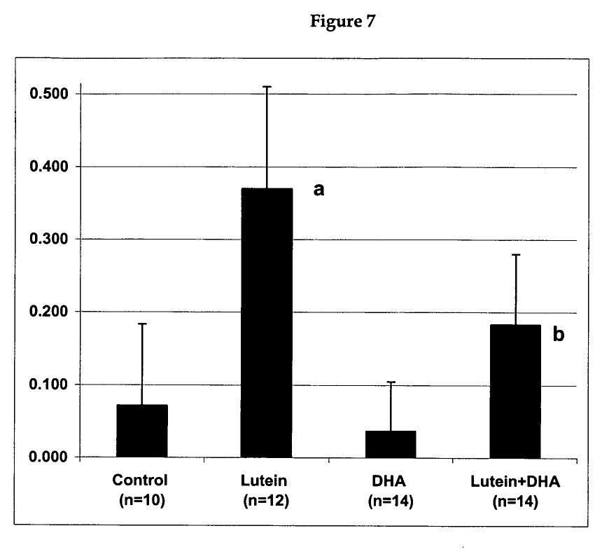

Total Macular Pigment Optical Density. The total MPOD significantly increased after lutein supplementation for 4 months (with and without DHA, p<0.035 and 0.015, respectively, FIG. 7). Although the mean increase in MPOD for the L group (0.370±0.140 OD) was greater that that for the LD group (0.183±0.097 OD), the difference was not significant due to the large variation in the data. No significant changes from baseline in MPOD were observed for the C and D groups (0.71±0.111 and 0.037±0.068 OD, respectively) (FIG. 7).

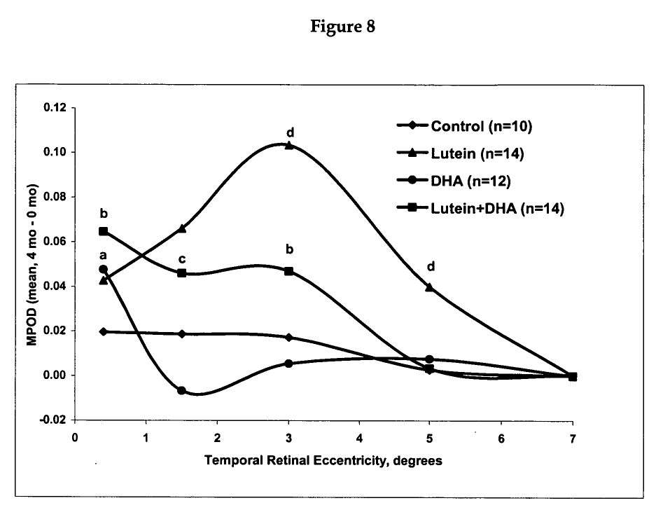

Distribution of MP. The distribution in the increases in MPOD varied with supplementation type. In the L group, there were increases in MPOD at each retinal loci, however, this was significant only at the 3° and 5° loci only (p<0.005, FIG. 8). In the D group, there was a significant increase in the most central locus (0.4°) (p<0.03) with no significant changes in the other retinal loci (FIG. 8). In the combination group (LD) the increase in MPOD was significant at the 0.4°, 1.5°, and 3° loci (p<0.05, FIG. 8).

MP Responders and Non Responders. Two of 14 in the L group and three of 14 subjects in the LD group did not have increases in MPOD with lutein supplementation. Response not related to BMI, age, baseline concentrations of serum lutein.

Serum Lipoproteins.

Lipoprotein size is importance in evaluating disease risk. Age-related macular degeneration and cardiovascular disease share many of the same risk factors. A common lipoprotein profile designated atherogeneic lipoprotein phenotype is characterized by a predominance of small dense LDL particles. Multiple features of this phenotype, including increased levels of triglyceride rich lipoprotein remnants and LDLs, reduced levels of HDL and an association with insulin resistance, contribute to increased risk for coronary heart disease compared with individuals with a predominance of larger LDL. Increased atherogenic potential of small dense LDL is suggested by greater propensity for transport into the subendothelial space, increased binding to arterial proteoglycans, and susceptibility to oxidative modification. Large LDL exhibits reduced LDL receptor affinity compared with intermediated sized LDL.

The Stanford Five City Project presented data indicating that LDL size is the strongest physiologic risk factor in conditional logistic regression analysis and is independent of HDL cholesterol, non-HDL cholesterol, and nonfasting triglycerides but not of the ratio total cholesterol/HDL cholesterol. The Quebec cardiovascular study reported that LDL particle size is an independent predictor of cardiovascular events that is independent of total cholesterol, LDL cholesterol, HDL cholesterol, triglycerides, total cholesterol/HDL cholesterol, apo B, and body mass index.

As shown below, the DHA and lutein (DL) group had the highest levels of large HDL and large LDL and lowest amount of small LDL at study end. Accordingly, the co-administration of at least one carotenoid, such as lutein, and DHA can be used to improve the cholesterol profile thereby reducing the risk of atherosclerosis and/or slowing or preventing the development of atherosclerosis, and reducing risk of heart attacks and strokes. The invention can be used to reduce existing cholesterol plaques on the artery walls, and reduce formation of cholesterol plaques and reduce the risk of rupture of cholesterol plaques.

The concentrations of serum total cholesterol and lipoproteins after 4 months of supplementation of lutein and/or DHA and in the control subjects are shown in Table 3. For all lipoproteins and for total serum cholesterol, there were no differences among the groups.

TABLE 3
Concentrations (mg cholesterol/dL) of serum total cholesterol
and lipoproteins after 4 months of supplementation
with lutein and/or DHA (mean ± se).

| | Cholesterol | VLDL | LDL | HDL |
|---|---|---|---|---|
| Control (n = 10) | 194 ± 8 | 92 ± 14 | 110 ± 6 | 66 ± 3 |
| DHA (n = 14) | 209 ± 5 | 78 ± 14 | 125 ± 5 | 67 ± 3 |
| Lutein (n = 12) | 199 ± 8 | 75 ± 12 | 113 ± 6 | 70 ± 5 |
| Lutein + DHA (n = 14) | 201 ± 8 | 50 ± 7 | 116 ± 8 | 73 ± 3 |

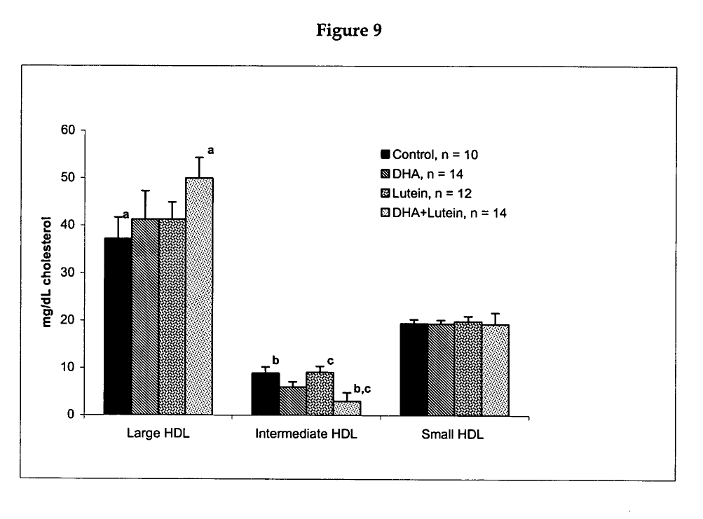

When the individual lipoprotein subfractions were examined, the LD group tended to have the largest differences from the C group. For the HDL subclasses, the large HDL subclass was significantly greater (p<0.013) and the intermediate HDL was significantly less in the LD group than the levels in the C group (p<0.025) (FIG. 9). Also, the intermediate HDL was significantly greater in the L group than in the LD group (p<0.010). There were no other differences among the groups in the HDL subclasses. For all groups, the large HDL subclass had the greatest concentrations.

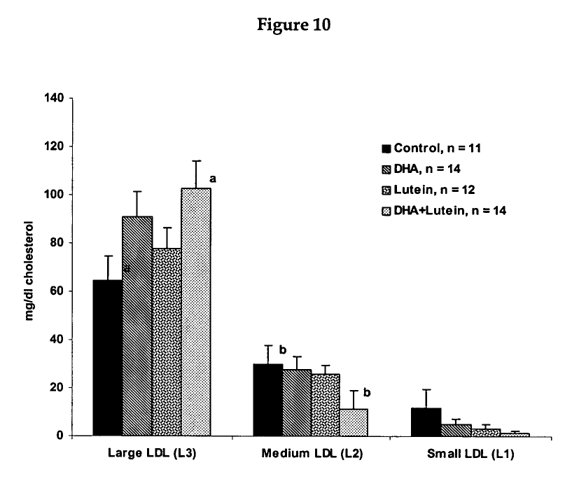

For the LDL subclasses, the large LDL subclass was significantly greater in the LD group than in the control group (p<0.006). The intermediate and small LDL were less in the LD group compared to the controls. However, this was only significant for the intermediate LDL (p<0.014) (FIG. 10). There were not other differences among the groups in the LDL subclasses. For all groups, the large LDL subclass had the greatest concentrations.

For the VLDL subclasses, the intermediate VLDL subclass was significantly greater (p<0.035) in the LD group than in the C group (FIG. 10). There was a tendency for the large VLDL to be less as well. However, this did not reach significance because of the large variability in the data (FIG. 10). There were not other differences among the groups in the VLDL subclasses. For all groups, the intermediate VLDL subclass had the greatest concentrations.

### Example 3

### Relations Between Serum Nutrient Levels and Cognitive Performance

Table 4 shows correlations between final test scores and possible covariates (age and education), DHA and lutein serum levels, and an endpoint, macular pigment ocular density (MPOD), from the primary vision study. Of the test scores that changed significantly after supplementation, Verbal Fluency and Trials to Learn Shopping List scores were the least subject to ceiling effects in the total sample of subjects. In the full sample (n=49), of age and education, age was the only possible covariate that was significantly associated with final Verbal Fluency score. Although subjects' scores on the Verbal Fluency test at baseline did not differ significantly by age, younger subjects recalled more instances of a category than older subjects at the end of the study.

TABLE 4
Correlations Between Variables After Supplementation.

| | | | | MIR |
|---|---|---|---|---|
| | | Shopping | Word List | Apartment |
| | Verbal | List (Trials | (Trials | Delayed |
| | Fluency | to Learn) | to Learn) | Recall |
| Age (N = 49) | −0.37** | 0.20 | 0.07 | −0.23* |
| Education | 0.11 | −0.18 | −0.19 | 0.05 |
| (N = 49) | | | | |
| DHA serum | 0.24* | −0.26* | −0.04 | 0.21 |
| (N = 49) | | | | |
| Lutein serum | 0.03 | 0.30** | −0.04 | −0.16 |
| (N = 48) | | | | |
| Lutein serum | 0.03 | 0.36** | −0.02 | −0.13 |
| (log-transformed | | | | |
| (N = 48) | | | | |
| Macular pigment | −0.09 | 0.32** | 0.13 | −0.32** |
| ocular density | | | | |
| (N = 48) | | | | |

*p <= 0.10
**p < 0.05

There was a trend toward a significant relationship between DHA serum levels and Verbal Fluency scores after supplementation. After adjustment for age, DHA serum level was significantly related to Verbal Fluency score (p=0.04). There was also a trend toward a significant relationship between DHA serum levels and Trials to Learn Shopping List scores, with higher DHA serum levels associated with learning the list in less trials.

Because the variable lutein serum level at end of study was highly positively skewed, the variable was log-transformed to produce a more normal distribution. No significant relationship was found between final lutein serum levels, with or without log-transformation, and Verbal Fluency scores. Also, in juxtaposition to the findings for DHA, higher lutein serum levels were significantly associated with needing more trials to learn shopping lists. This result can be understood by looking at FIG. 2, which shows that subjects in the Lutein supplementation group had some of the highest scores for the variable Trials to Learn Shopping List.

In the vision study, final lutein serum levels in the total subject sample were marginally associated with MPOD after supplementation (r=0.27, P=0.07). It is interesting to note that MPOD was significantly related to two cognitive test scores, but in the opposite direction than would be expected if higher MPOD were associated positively with better cognitive functioning in this study. After supplementation, subjects with higher MPOD required more trials to learn the shopping list and recalled less items in the MIR Apartment Delayed Recall task.

Despite the small numbers of subjects and restricted initial ranges of scores for many of the cognitive tests because of subjects' competence, results in this study suggest that DHA and lutein act synergistically to improving cognitive performance. In this study, supplementation with both DHA and lutein was associated with significant results or a trend toward a significant result on several cognitive tests. Each of these tests required subjects to retrieve or learn and retrieve information from memory in a time-limited or efficient fashion. In the Verbal Fluency test, subjects had to recall instances of a category in a short period of time. In the Shopping List and Word List tasks, subjects were asked to learn all items presented in lists verbally or on a computer screen over trials. Although subject groups did not increase the number of items they recalled on the first trial of either test, the DHA and lutein supplementation group learned lists with fewer trials on average after supplementation. In the MIR Apartment test, which required subjects to remember objects after only one learning opportunity but with control of how they organized and remembered items, subjects in the DHA and lutein supplementation group recalled significantly more objects after supplementation, although they did not increase items recalled after a delay on other memory tests in which items were presented to them at a constant rate by interviewer or on a computer monitor.

On the Verbal Fluency test, subject groups who had been supplemented with either DHA or lutein also showed significant improvement. Because this test evoked one of the least restricted ranges of scores in this subject sample, there is reason to believe that further studies might elicit improvements in cognitive status with either nutrient alone, given subject samples with more variability and possibly tests with similar characteristics. In particular, DHA supplementation might be better assessed in a subject group with scores less close to ceiling; as shown in the Figures and Table 2, the DHA supplementation group had less room to improve than the other treatment groups.

Generally the subjects in this study, although elderly, were competent at the tests, and some of the oldest subjects were among the most competent initially. On average, control subjects appear to have been among the strongest performers at baseline on span measures of memory, and similar to the DHA supplementation group, initially high scores might have reduced their ability to improve on some cognitive measures. However, control subject scores were not among the highest at baseline on the Verbal Fluency test. The lack of significant change on the Verbal Fluency test by control group subjects suggests that lack of supplementation was likely associated with lack of improvement.

TABLE 5
Effect of Lutein and DHA supplementation of Cognitive Function
(Significance of 4 month change from baseline).

| | Control | Lutein | DHA | Lutein + DHA |
|---|---|---|---|---|
| Verbal Fluency | NS | 0.00 | 0.03 | 0.00 |
| Memory rate of | NS | NS | NS | 0.07 |
| learning | | | | |
| Shopping list | NS | NS | NS | 0.03 |
| memory test | | | | |
| Word list | NS | NS | NS | 0.07 |
| memory test | | | | |
| Apartment | NS | NS | NS | 0.02 |
| memory test | | | | |

Also, the significant association between serum levels of DHA and Verbal Fluency scores after 4 months (summarized in Table 5) of supplementation identifies a possible mechanism by which cognitive improvement in many of the subjects may have occurred. It should be noted that a similar relationship between final serum levels of lutein and Verbal Fluency scores was not found. There was also no significant relationship found between final Verbal Fluency scores and macular pigment ocular density, a significant dependent variable in the primary study of vision. The association of serum lutein levels with MPOD after supplementation provides strong corroboration that serum lutein increased MPOD. Lutein supplementation was associated with improvement in Verbal Fluency scores. As in the macula, among the carotenoids, there is a preference for lutein to accumulate in the brain. A facilitation of lutein uptake into the brain by DHA (via increases in HDL subfractions) may occur.

### Example 4

### Relations Between Serum Nutrient Levels and MPOD

The results of this study demonstrate that supplementation with daily oral doses of lutein (12 mg/d for 4 months) is effective in increasing circulating levels of lutein as well as MPOD. About ⅓ of subjects in L and LD groups did not have increases in MPOD with lutein supplementation. A MPOD response was not related to BMI, age, dietary intake of lutein or baseline serum and macular concentrations of lutein.

The invention is based, in part, on the unexpected effects of DHA supplementation on serum lutein and MPOD. The increases from baseline in serum lutein at 2 month for the LD group were greater than that for the group supplemented with lutein alone (L group). Significant increases were found in the central macula (0.4° locus) in the D and and a trend towards an increase in the LD group, but not the L group. Interestingly, the effects of lutein+DHA supplementation on the MPOD appeared to be a combination of the individual effects of lutein and DHA supplementation. Thus, supplementation with DHA can provide beneficial effects on the circulating concentrations of lutein.

The mechanism by which DHA increases MPOD may be due to its effects on the transport and uptake of lutein into the macula. Although there were no significant differences in the serum lipoproteins among the groups, there were differences in the lipoprotein subclasses. Evaluation of serum lipoprotein subclasses has been suggested as a useful tool in assessing the risk of cardiovascular disease. The effects of DHA (fish oil) on these subclasses are towards an improve lipoprotein profile. Given that cardiovascular disease and AMD share some for the same risk factors, these changes may be useful for a decreased risk of AMD. Indeed, DHA status has been related to a decreased risk of AMD. In this example, cross-sectional changes in the lipoprotein subclasses among the four groups were measured. In most cases, the LD group had the greatest differences from controls, these changes being of towards a less atherogenic pattern. Given that these changes would affect transport and delivery of lipids to tissue, e.g. xanthophylls to the macula, it is tempting to speculate that the increases in MPOD in the D group were due to a change in lipoprotein profile that promotes the uptake of xanthophyll into the retina. These effects appear to occur centrally in the macula given the significant increases in both the D and LD groups that did not occur for the C or L groups. The reason for the preference towards an accumulation that is central, rather than eccentric, is not known. Given that zeaxanthin rather than lutein accumulates preferentially in the central macula, perhaps the effects of DHA are specific to this xanthophyll.

In conclusion, supplementation of these women with lutein alone increased MPOD eccentrically whereas DHA supplementation alone resulted in central increases in MPOD. The combination of supplements had a combined effect on increasing MPOD. DHA facilitated accumulation of lutein in the blood and macula. These effects may have occurred through DHA altering the lipoprotein profile.

The results indicate that carotenoid supplementation in combination with DHA supplementation can effectively increase absorption of carotenoids in the blood and macula. The co-administration of lutein and DHA was found to have a neuroprotective effect, leading to increased memory and cognitive function. In addition, the co-administration was shown to alter the lipoprotein profile, resulting in increase lutein concentration in the macula. Thus, the combination of supplemental lutein and DHA can be used to prevent or slow the progression of AMD. The study confirms that oral administration of the composition of the present invention is effective as a nutritional supplement, either therapeutically or prophylactically.

While the present invention has been described in terms of specific methods and compositions, it is understood that variations and modifications will occur to those skilled in the art upon consideration of the present invention. Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims. All publications and references are herein expressly incorporated by reference in their entirety.

1. A method for modulating macular pigmentation by enhancing carotenoid absorption in a subject comprising
co-administering synergistic amounts of at least one carotenoid and docosahexanaenoic acid (DHA) to a subject, such that the administration produces increased absorption of the carotenoid.

2. The method of claim 1, wherein the at least one carotenoid is selected from the group consisting of lutein and zeaxanthin.

3. The method of claim 1, wherein the at least one carotenoid is lutein.

4. The method of claim 3, wherein the method comprises administering 500 to 1500 mg/day of DHA and about 6 to 24 mg/day of lutein.

5. The method of claim 3, wherein the method further comprises administering about 700 to 1000 mg/day of DHA and about 10 to 15 mg/day of lutein.

6. The method of claim 3, wherein said co-administration increases serum carotenoid concentration levels between about 100 nmol/L to about 1 μmol/L compared to serum carotenoid concentrations levels in the absence of DHA.

7. The method of claim 1, wherein the administration produces an increase in retinal concentration of lutein.

8. A method increasing transport of lutein into a subject's retina,
administering DHA in sufficient quantity to increase HDL and HDL subfractions of blood,
administering lutein, wherein the co-administration of DHA increases macular pigment (MP) via increased transport of lutein into the retina.

9. The method of claim 8, wherein the method comprises decreasing macular degeneration.
